(19) United States
(12) Patent Application Publication
(10) Pub. No.: US 2025/0179212 A1
(43) Pub. Date: Jun. 5, 2025

(54) METHODS OF TREATING CANCER AND MONITORING CANCER PROGRESSION

(71) Applicant: The University of British Columbia, Vancouver (CA)

(72) Inventors: Wilfred A. Jefferies, Surrey (CA); Wenjing Xia, Surrey (CA)

(21) Appl. No.: 18/847,328

(22) PCT Filed: Jan. 9, 2023
(86) PCT No.: PCT/CA2023/050013

§ 371 Date: Sep. 16, 2024

Related U.S. Application Data
(60) Provisional application No. 63/297,923, filed on Jan. 10, 2022.

(51) Int. Cl. C07K 16/30; A61P 35/00; C07K 16/18; C12N 15/11; C12N 15/115

(52) U.S. Cl. CPC C07K 16/3092; A61P 35/00; C07K 16/18; C12N 15/11; C12N 15/115

(57) ABSTRACT

The present invention provides methods of treating cancer by modulating regulators of IL-33. Also provided are methods of preventing tumor metastasis and/or cancer progression by treatment with agents that modulate FOX1A. Also provided are diagnostic methods for assessing cancer prognosis.

### FIELD OF THE INVENTION

The present invention relates to the field of cancer therapeutics and monitoring cancer progression. In particular, the present invention relates to modulation of cancer immunity and a method of monitoring for tumor transition to metastasis.

### BACKGROUND OF THE INVENTION

Understanding the mechanisms that promote a primary cancer to advance to a metastatic derivative is of great concern as metastatic cancers account for 90% of all cancer deaths. Recent advances in immunotherapies include the use of adoptive transfer of autologous T cells, or chimeric antigen receptor (CAR)-modified T cells, tumour antigen vaccines, dendritic cell based vaccines, checkpoint blockade inhibitors against CTLA-4, PD-L1 and PD-1, T cell agonistic antibodies such as OX40, and 4-1BB and oncolytic viruses driving expression of GM-CSF. However, tumour heterogeneity remains a major obstacle to the development of effective therapies. Genetic and epigenetic instability and alterations, as well as changing tumour microenvironments, results in tumours composed of diverse subclones, with varying genetic and phenotypic characteristics. Furthermore, intra-tumour heterogeneity may enable clonal cooperation resulting in enhanced tumour progression. Thus, in this neo-Darwinian process, the tumour microenvironment can contribute to cancer evolution. Specific aspects of the tumour microenvironment that may provide selective pressures to evolve new cancers' phenotypes include: resource and metabolite limitations, and deleterious conditions such as tissue hypoxia, acidosis, and importantly, selection by cancer therapeutics and immune-surveillance. The selective pressure of immune-surveillance on genetically unstable tumour populations may yield tumours that have lost expression of antigen processing machinery (APM) components, often resulting in reduced assembly of functional major histocompatibility complex (MHC or HLA) molecules. The mechanism undelying immunoevasion of the adaptive immune system was first described by Alimonte et al. (2000) and termed immune-subversion or immune-escape and subsequently confirmed by Shankaran et al., 2001; https://www.nature.com/articles/35074122) and termed immune-editing. The loss of APM components and functional MHC-I (HLA-I) molecules with an immune-escape may occur in up to 90% of patients and is associated with tumour aggressiveness and increased metastatic potential. Several types of cancer, including breast cancer, renal carcinoma, melanoma, colorectal carcinoma, head and neck squamous cell cancer, cervical cancer, and prostate carcinoma show a clear correlation between HLA down-regulation and poor prognosis. Furthermore, tumours that become ‘invisible’ or unrecognizable by cytolytic T-cells (CTLs) and may also become refractory to emerging immunotherapeutics such as CAR-T cells and immune checkpoint blockage inhibitors. Currently only 15-30% of patients respond to current immunotherapies. Thus, immunotherapy has improved outcomes for some patients, but most remain unresponsive and selecting cancer patients who may respond positively to immunotherapies remains a challenge and discovering new and better treatment options and selecting patients that respond to emerging immunotherapy modalities is a priority.

It has been previously demonstrated that by restoring TAP-1 expression in metastatic cells it is possible to restore APM and the CTL recognition of MHC-I molecules in murine carcinomas. Additionally, it was shown that APM deficiency can be restored in vitro and in vivo by complementation of TAP expression, by either transformation with virus vectors containing the TAP gene or with immune enhancers. Intriguingly, in a previous study it has been found the TAP-1 deficiency was not regulated by defects or mutations in the TAP-1 gene, but it was epigenetically regulated and could be restored by treatment with histone deacetylase inhibitors (HDACi), such as trichostatin-A (TSA). The increasing frequency of immune-escape tumour variants in many forms of metastatic cancers is a predictor of disease progression as well as patient outcome. However, few attempts have been made to directly identify genetic mutations that drive APM deficits in immune-escape tumour variants.

It was recently discovered a new mechanism of immune-escape and demonstrated that metastatic forms of prostate and renal carcinomas express reduced levels of the ‘alarmin’ interleukin 33 (IL-33), and furthermore, that low levels of IL-33 serve as an immune biomarker for cancer reoccurrence and clinical outcome. IL-33 controls a regulatory loop that maintains MHC-I expression in normal epithelium and primary tumours, a process that is required for immune-surveillance by the adaptive immune response. The absence of IL-33 in metastatic prostate tumours allows these cells to undermine recognition by T cells and to subvert the host immune response. This mechanism of immune-escape is common in several solid tumours, and therefore is a major obstacle to actuating effective immunotherapies. It was subsequently demonstrated that epigenetic silencing of APM components can result in immune-escape and can be reversed by histone deacetylase inhibitor (HDACi) or cytokines such as interferon gamma and IL-33. It was also demonstrated that expression of the IL-33 transgene in metastatic tumours, or treating tumour-bearing mice with the IL-33 cytokine, boosts immune responses and reducing tumour growth in vivo. Regulatory genes in the APP pathways remain largely unexplained. Regulatory genes in the APP pathways may be novel targets for cancer drug development.

### SUMMARY OF THE INVENTION

An object of the present invention is methods of treating cancer and monitoring cancer progression. In accordance with an aspect of the present invention, there is provided a method of inhibiting cancer progression and/or treating of cancer, said method comprising enhancing expression and/or activity of interleukin-33 and/or APP by modulating one or more regulators of interleukin-33 and/or APP.

In accordance with an aspect of the present invention, there is provided a method of stimulating antigen presentation, said method comprising modulating expression and/or activity of one or more regulators of interleukin-33 and/or APP.

In accordance with an aspect of the present invention, there is provided a method of inhibiting metastatic spread of circulating tumor cells to distal organs, said method comprising enhancing expression and/or activity of interleukin-33 and/or APP by modulating one or more regulators of interleukin-33 and/or APP.

In certain embodiments, the regulator is a negative regulator and said method comprises decreasing expression and or activity of said negative regulator. In certain embodiments, the regulator is FOXA1.

In certain embodiments, the method comprises administration of an agent that targets said regulator and inhibits activity of said regulator. In certain embodiments, the agent is an antisense, antibody, aptamer or small molecule

In certain embodiments, the agent is used in combination with other therapies which stimulate immunity, treat cancer and/or inhibit cancer progression.

In accordance with an aspect of the present invention, there is provided a method of distinguishing metastatic cells from non-metastatic cells, said method comprising by determining expression and/or mutations of one or more master regulators involved in the IL33 and/or APP.

In accordance with an aspect of the present invention, there is provided a method for determining progression to metastatic disease, said method comprising determining expression and/or mutations of one or more master regulators involved in the IL33 and/or APP.

In accordance with an aspect of the present invention, there is provided a method of determining prognosis of cancer by determining expression and/or mutations of one or more master regulators involved in the IL33 and/or APP. In certain embodiments, the one or more master regulator is FOX1A.

### BRIEF DESCRIPTION OF THE FIGURES

FIG. 1 illustrates that HLA expression is correlated to the level of IL-33. HLA expression is co-regulated with IL-33 levels in human prostate cancer. Expression levels of mRNA of IL-33, CD8a, CD4 (left panel) and HLA-A, HLA-B, HLA-C (right panel) in castration-resistant prostate cancer (CRPC) are low relative to benign prostate tissue and both low- and high-risk primary tumours. D'Amico Risk classification: low-risk=prostate-specific antigen (PSA)≤10, Gleason score≤6, and clinical stage T1-2a; high-risk=PSA>20, Gleason score ≥8, or clinical stage T2c-3a. *P<0.05 (Student's t-test) when comparing high-risk primary (treated) tumours and metastatic CRPC.

FIG. 2 illustrates correlation between IL-33 and immune cell expression in different forms of prostate cancer. Classification is based on clinical representation of the disease. Ben=benign; Low=low-risk primary tumours; High=high-risk primary tumours; CRPC=castration-resistant prostate cancer.

FIG. 3 illustrates a sample flow chart of bioinformatic screening process.

FIG. 4 illustrates a sample flow chart of gene identification and in vitro characterization. A) FOXA1 mutations appear more frequently in low-IL33 expressing tumour samples than in high-IL33 expressing tumour samples. B) Target gene (e.g. FOXA1) will either be knocked-out or overexpressed in tumour cells, and then resulting MHC-I surface protein expression will be determined, as will their ability to be recognized and killed by cytotoxic T cells.

FIG. 5 illustrates CRISPR-Cas9 screening overview.

FIG. 6 illustrates sample flow chart of experimental validation for cancer cells containing gene knock-outs.

FIG. 7 illustrates that mutations occur around the functional domain of FOXA1.

FIG. 8 illustrates that expression IL33 positively correlates to APM genes, negatively correlates to FOXA1.

FIG. 9 illustrates FOXA1 potentially regulates IL33 by binding to enhancer region.

FIG. 10 illustrates that IL-33 gene-complementation suppresses tumour growth rate in vivo and inhibits metastatic spread of tumour cells in a mouse model. Metastatic A9 cells were derived from a primary mouse lung tumour, TC-1. (a) Stable transfection of IL-33 gene into metastatic A9 cells resulted in significantly inhibited tumour formation in mice; P<0.05, when comparing IL-33 expressing tumours (primary TC-1 or A9+ IL-33) to metastatic A9 alone (Student's t-test). (b) GFP-positive circulating tumour cells were detected in adrenal glands that were distal from initial subcutaneous inoculation, and assessed using flow cytometry. (c) Quantification of GFP-positive circulating tumour cells. The percentage of all cells was calculated from the fraction of live cells in 5×10⁵ events used to create a profile for each organ. The graph corresponds to the data from eight representative animals. *P<0.05, comparing GFP-positive circulating tumour cells isolated from the primary (TC1) and metastatic (A9) bearing animals (Student's t-test).

FIG. 11 illustrates IL-33-gene complementation of immune evasive tumours shows clear phenotypic shift towards immune recognition.

FIG. 12 illustrates reduced IL-33 expression is associated with prostate cancer progression (human study, IL-33 protein expression: tissue microarray (VPC)).

FIG. 13 illustrates reduced IL-33 expression is associated with cancer progression (human study).

FIG. 14 illustrates more genome instability in IL33 low cohort.

FIG. 15 illustrates higher AR activity and PSA scores in low cohort.

FIG. 16 illustrates IL33 low cohort has a lymphocyte depleted immune subtype.

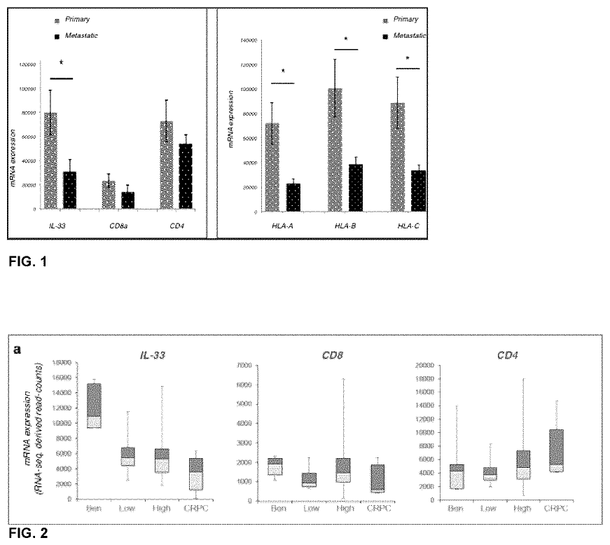

These and other features of the invention will become more apparent in the following detailed description in which reference is made to the appended drawings.

### DETAILED DESCRIPTION

The present invention is based on the identification of genes that potentially function as “master regulators” that regulate the interleukin-33 and antigen presentation pathway (APP) pathways. In particular, the present invention is based on the discovery of that FOXA1 is more frequently mutated in prostate adneocarcinomas that express less IL-33 and expression of IL-33 positively correlates to APM genes and negatively correlates to FOXA1. Interleukin 33 (IL-33) appears to play a role in cancer progression. IL-33 is down regulated or mutated in metastatic tumors compared to benign or primary tumors and the growth of metastatic tumors and the frequency of circulating metastatic tumor cells (CTC) are reduced when the tumors are genetically engineered to express IL-33.

Accordingly, the present invention provides methods and compositions for inhibiting cancer progression and/or for providing treatment of cancer by modulating one or more regulators of interleukin-33 and/or APP. In certain embodiments, the present invention provides methods and compositions for inhibiting cancer progression and/or for providing treatment of cancer by enhancing expression and/or activity of one or more positive regulators of interleukin-33 and/or APP. In certain embodiments, the present invention provides methods and compositions for inhibiting cancer progression and/or for providing treatment of cancer by decreasing expression and/or activity of one or more negative regulators of interleukin-33 and/or APP. In certain embodiments, the regulator is selected from the group consisting of FOXA1, KIF13A, MUC16, CSMD3, MUC17, ACACA, ATM, CTNNB1 and FBN3. In certain embodiments, the regulator is FOXA1.

Inhibition of cancer progression and/or cancer treatment includes but is not limited to inhibition of tumor growth, stimulation of tumor regression, enhancement of immune recognition of tumor cells, stimulation of anti-tumor immunity, treatment of the primary tumor, prevention and/or treatment of tumor metastases.

Also provided are methods of stimulating antigen presentation by modulating one or more regulators of interleukin-33 and/or APP. In certain embodiments, the present invention provides methods of stimulating antigen presentation by enhancing expression and/or activity of one or more positive regulators of interleukin-33 and/or APP. In certain embodiments, the present invention provides methods of stimulating antigen presentation by decreasing expression and/or activity of one or more negative regulators of interleukin-33 and/or APP. In certain embodiments, the regulator is selected from the group consisting of FOXA1, KIF13A, MUC16, CSMD3, MUC17, ACACA, ATM, CTNNB1 and FBN3. In certain embodiments, the regulator is FOXA1. The antigen(s) may be one or more cancer antigens or one or more antigens from one or more pathogens.

A worker skilled in the art could readily determine which types of cancers can be treated. The cancer may be a solid tumor. In certain embodiments, the cancer is a sarcoma, carcinoma or lymphoma. In certain embodiments the cancer is selected from a breast cancer, ovarian cancer, liver cancer, renal cancer, melanoma, colorectal cancer, head and neck squamous cell cancer, cervical cancer or prostate cancer. In certain embodiments, the cancer is a leukemia or other hematological cancers.

In certain embodiments, there is provided methods and compositions for inhibition of cancer progression and/or treatment of cancer by modulating expression of one or more regulators of IL-33 and/or APP. In certain embodiments, the regulator(s) are selected from the group consisting of FOXA1, KIF13A, MUC16, CSMD3, MUC17, ACACA, ATM, CTNNB1 and FBN3. In certain embodiments, the regulator is FOXA1.

In certain embodiments, there is provided a method of inhibiting metastatic spread of circulating tumor cells to distal organs by modulating expression of one or more regulator(s) of IL-33 and/or APP. In certain embodiments, the one or more regulator(s) is selected from the group consisting of FOXA1, KIF13A, MUC16, CSMD3, MUC17, ACACA, ATM, CTNNB1 and FBN3. In certain embodiments, the regulator is FOXA1.

The modulators of regulator(s) of IL-33 and/or APP may be used alone or in combination with each other and/or with other therapies which stimulate immunity, treat cancer and/or inhibit cancer progression. Other therapies include but are not limited to cytokines, cellular therapies (including but not limited to administration of immune cells such as ILC2s), vaccine therapies, and chemotherapeutics.

Specific non-limiting examples of therapies that may be used in combination include but are not limited TNF alpha; interleukin-33; interleukin-21; interleukin-13; a combination of interleukin (IL)-4, IL-5, IL-9 and IL-13; a combination of PD-1, CTLA-4, PDL-1; interferon, including but not limited to interferon alpha, beta or gamma; GM-CSF; G-CSF; HDACi; HATs; methylation inhibitors; T cells including but not limited to CAR T Cells, autologous T Cells, autologous T Cells transducer with specific TCRs, autologous B Cells, dendritic cells subsets, antigens of interest including but not limited to viral, bacterial and tumor antigens; antibodies including but not limited to antibodies targeting tumor antigens such herceptin; other biological therapies; hematopoietic stem-cell transplantation; natural killer cells; Toll receptor agonists; chemokines; anti-angiogenic molecules; other cytokines used in immune therapy including but not limited to IL-2; chemotherapies; viral vectors; oncolytic viruses; adjuvants; cytotoxic agents; and therapies which deplete regulatory T cells.

In certain embodiments, there is provided a method of modulating immunity and/or an immune response by modulating expression of IL-33 by modulating expression and/or activity of one or more regulator(s) of IL-33. In certain embodiments, there is provided a method of modulating antigen presentation by modulating expression of IL-33 by modulating expression and/or activity of a regulator of IL-33. In certain embodiments, there is provided a method of modulating MHCI expression by modulating expression modulating expression of IL-33 by modulating expression and/or activity of one or more regulators of IL-33. In certain embodiments, the one or more regulator(s) are selected from the group consisting of FOXA1, KIF13A, MUC16, CSMD3, MUC17, ACACA, ATM, CTNNB1 and FBN3. In certain embodiments, the regulator is FOXA1.

In certain embodiments there is provided a method of enhancing immunity and/or an immune response by enhancing expression of IL-33 by modulating expression and/or activity of regulator of IL-33. In certain embodiments, there is provided a method of enhancing antigen presentation by enhancing expression of IL-33 by modulating expression and/or activity of FOXA1. In certain embodiments, there is provided a method of enhancing MHCI expression by modulating expression of IL-33 by modulating expression and/or activity of one or more regulators of IL-33. In certain embodiments, the one or more regulators are selected from the group consisting of FOXA1, KIF13A, MUC16, CSMD3, MUC17, ACACA, ATM, CTNNB1 and FBN3. In certain embodiments, the regulator is FOXA1. Non-limiting examples of methods to enhance expression and/or activity a polypeptide interest include administration of the polypeptide of interest, administration of a nucleic acid or vector which encodes the polypeptide of interest or administration of one or more molecules which enhance expression of the polypeptide of interest.

In certain embodiments, the methods may be used to enhance an anti-cancer immune response or an anti-pathogen immune response. The methods may be used alone or in combination with other therapies.

In certain embodiments, one or more modulators of IL-33 and/or the APP pathway may be used as adjuvants.

In alternate embodiments, there is provided a method of decreasing immunity and/or an immune response by inhibiting expression and/or activity of IL-33 by modulating expression and/or activity of a regulator of IL-33. In certain embodiments, the regulator is selected from the group consisting of FOXA1, KIF13A, MUC16, CSMD3, MUC17, ACACA, ATM, CTNNB1 and FBN3. In certain embodiments, the regulator is FOXA1. In certain alternate embodiments, there is provided a method of decreasing antigen presentation by inhibiting expression and/or activity of IL-33 by modulating expression and/or activity of a regulator of IL-33. In certain embodiments, the regulator is selected from the group consisting of FOXA1, KIF13A, MUC16, CSMD3, MUC17, ACACA, ATM, CTNNB1 and FBN3. In certain embodiments, the regulator is FOXA1.

### Diagnostic Methods

It is hypothesized that master regulators involved in the IL33 and APP will be more frequently mutated in low-IL33 expressing tumours than tumours expressing high levels of IL33. As demonstrated herein, FOXA1 is more frequently mutated in low-IL33 expressing tumours than tumours expressing high levels of IL33. The ability to escape from immune surveillance is a hallmark of cancer. Primary tumour progression is controlled by the host immune surveillance until driver mutations evolve mechanisms to escape. Decrease in IL33 has been shown to be an early immune biomarker for prostate tumour transition to its metastatic form, and it is closely associated with important antigen processing and presentation (APP) pathways.

Accordingly, in certain embodiments, there is provided a method of distinguishing metastatic cells from non-metastatic cells by determining expression and/or mutations of one or more master regulators involved in the IL33 and/or APP. In other embodiments, there is provided a method for determining progression to metastatic disease by determining expression and/or mutations of one or more master regulators involved in the IL33 and/or APP. In certain embodiments, there is provided a method of determining prognosis of cancer by determining expression and/or mutations of one or more master regulators involved in the IL33 and/or APP. In specific embodiments, the one or more master regulator is FOX1A.

In certain embodiments, there is provided a method for determining progression to metastatic disease by determining expression of IL-33, various APP genes (such as MR1) and/or mutations or expression of one or more regulators of IL-33 including but not limited to FOX1A. In some embodiments, there is provided a method of diagnosing progression to a metastatic form of prostate cancer or metastatic lung cancer by determining level of level of expression of FOX1A or mutations of FOX1A. In certain embodiments, expression of IL-33 is also determined.

In certain embodiments, there is provided a method of determining clinical outcome of a cancer patient by determining FOX1A mutations and/or expression level of IL-33. In specific embodiments, mutations and/or expression of KIF13A and/or CTNNB1 is also determined. In other embodiments, patient status is monitored other time by monitoring changes in expression of IL-33 over time. In certain embodiments, changes in expression of IL-33 and/or number of mutations in FOX1A are monitored over time.

In certain embodiments, there is provided a method of identifying regulatory network of IL33 in cancer, said method comprising determining genes mutated or expression of genes in cancer cells which have high IL33 expression and cancer cells which have low IL33 expression.

To gain a better understanding of the invention described herein, the following examples are set forth. It will be understood that these examples are intended to describe illustrative embodiments of the invention and are not intended to limit the scope of the invention in any way.

### Example 1

Hypothesis: Master regulators involved in the IL33 and APP are more frequently mutated in low-IL33 expressing prostate tumours than tumours expressing high levels of IL33.

### Methods

i) Bioinformatic analysis: The Cancer Genome Atlas (TCGA, Cancer Genome Atlas Research Network) contains genomic, expression and clinical data from cancer patients, including various forms and stages of prostate cancer. Furthermore, the Vancouver Prostate Centre has an in-house databank collated from patient prostate tumours. For the study, gene expression data from prostate samples will first be segregated into cohorts using two strategies: 1) different clinical outcomes based on disease state (primary vs. metastatic); 2) IL33-high vs IL33-low cohorts based on expression data. Frequently mutated genes in the metastatic clinical outcome and IL33-low cohorts will be identified. Within each cohort, genes that are recurrently mutated will be identified and ranked based on mutation frequencies and molecular functions. For example, if any mutation is accountable for the downregulation of IL33, it should appear more frequently in the IL33 low cohorts, with functional impacts (see FIG. 3 for sample flow chart). It is expected that good gene candidates will include: positive acting transcription factors and/or regulators of epigenetic programs.

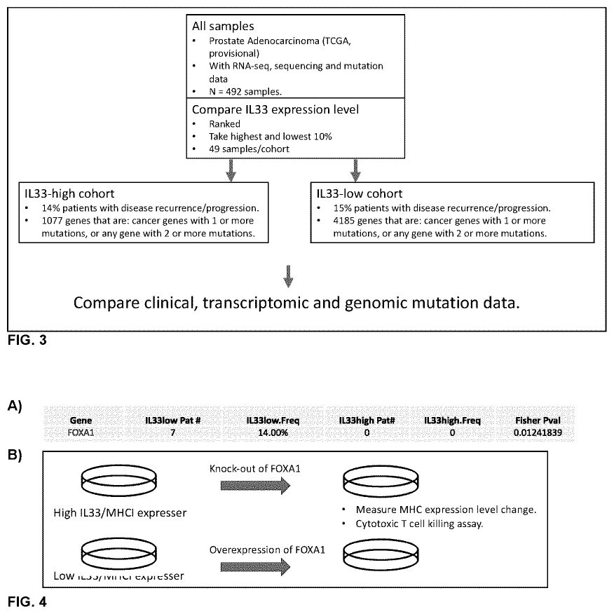

Validation of identified genes: Gene will be validated for their ability to regulate cancer pathways using in-vitro and in-vivo models.
a) In vitro models: Prostate cancer cell lines will be characterized using various assays, including: RT-PCR and qPCR to validate gene expression; Western blot to validate protein expression levels; flow cytometry to confirm high/low expression of IL33 and MHC-I. Gene manipulation will be used to both knock-out or overexpress the top hit genes, e.g. FOXA1, within appropriate cell lines, and determine whether the cells express MHC-I and are more recognizable to cytotoxic T cells (see FIG. 4 for sample flow chart).
b) In vivo models: The most promising gene will be tested in a mouse tumour model. The gene identified as a regulator will be expressed or knocked-out within a prostate cell line and then the ability of those cells to form a tumour in a mouse will be monitored.

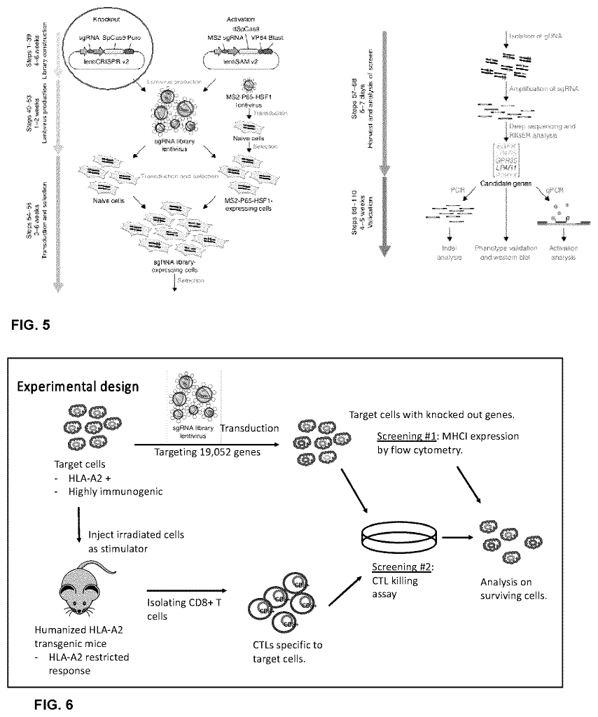

Hypothesis: Mutation in genes that are important for prostate cancer antigenicity and immunogenicity will disable the recognition and killing of cancer cells by immune effector cells.
i) CRISPR-Cas9 Knock-out: Human cancer cell lines with high antigenicity will be genomically transduced with CRISPR-Cas9 knockout system (GECKO library, composed of 18,080 genes) (see FIG. 5 for outline of lentiviral CRISPR-Cas9 system). Note, the cancer cells used will express HLA-A2 for use in the CTL assay below.
ii) Gene knockout screening: The transduced cells will be screened by two methods (see FIG. 6 for overall sample flow chart):
a) MHCI expression decrease: Genes whose loss-of-function decreases the surface expression of MHCI as seen through using flow cytometry will be screened.
b) Survival of cancer cells in a CTL killing assay: Genes whose loss-of-function enables the survival of cancer cells from antigen-specific killing by CTLs will be screened. HLA-A2402-Kb mice will be initially inoculated with HLA-A2-restricted human cancer cells. After 14 days, the mice will be sacrificed, blood will be collected and CD8⁺ cytotoxic T cells (CTL) expressing HLA-A2 will be collected for use in the CTL killing assay against the cancer cells.

### Results

Clinical correlations of changes in IL33 between Normal
tissue and primary lesions in different tumour types

| Cancer type | Patients | Log2_fold_change | p value |
|---|---|---|---|
| Cervical cancer/ | 3/3(100.0) | −5.75 | 9.66E−16 |
| Cervical squamous | | | |
| cell carcinoma/ | | | |
| Endocervical | | | |
| adenocarcinoma | | | |
| Urinary bladder | 19/19(100.0) | −2.99 | 1.48E−12 |
| cancer | | | |
| Esophageal cancer | 9/11(81.82) | −2.82 | 1.13E−06 |
| Lung cancer/Lung | 49/51(96.08) | −2.25 | 3.71E−15 |
| squamous cell | | | |
| carcinoma | | | |
| Head and neck cancer | 37/43(86.05) | −2.19 | 4.96E−13 |
| Uterine cancer | 6/7(85.71) | −2.18 | 5.58E−03 |
| Breast cancer | 59/114(51.75) | −1.3 | 4.14E−07 |
| Prostate cancer | 42/52(80.77) | −0.92 | 2.60E−09 |
| Thyroid cancer | 51/59(86.44) | −0.66 | 4.27E−03 |

Wan, Quan et al. “BioXpress: an integrated RNA-seq-derived gene expression database for pan-cancer analysis.” Database: the journal of biological databases and curation (2015)

FOXA1 is more frequently mutated in IL-33 low cohort of prostate adenocarcinoma.

| | IL33low | IL33low. | IL33high | IL33high. | Fisher |
|---|---|---|---|---|---|
| Gene | Pat # | Freq | Pat# | Freq | Pval |
| FOXA1 | 7 | 14.00% | 0 | 0 | 0.01241 |
| KIF13A | 5 | 10% | 0 | 0 | 0.05615 |
| MUC16 | 5 | 10% | 0 | 0 | 0.05615 |
| CSMD3 | 6 | 12% | 1 | 2% | 0.11147 |
| MUC17 | 6 | 12% | 1 | 2% | 0.11147 |
| ACACA | 4 | 8% | 0 | 0 | 0.11730 |
| ATM | 4 | 8% | 0 | 0 | 0.11730 |
| CTNNB1 | 4 | 8% | 0 | 0 | 0.11730 |
| FBN3 | 4 | 8% | 0 | 0 | 0.11730 |

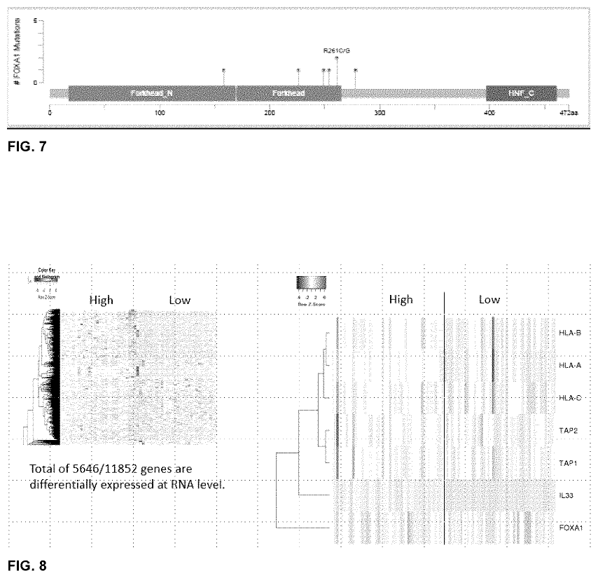

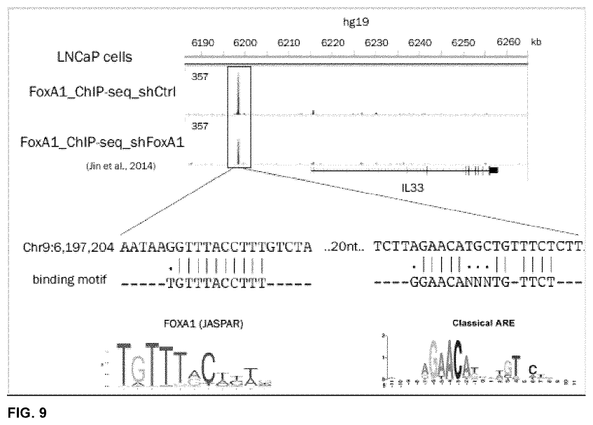

Mutations occur around the functional domain of FOXA1. See FIG. 7.

IL-33 gene-complementation inhibits metastatic spread of tumour cells to distal organs. GFP-positive circulating tumour cells were isolated from sites that were distal from initial subcutaneous inoculation and assessed using Flow Cytometry. Shown here are representative results from each of the four tumour groups, where n=eight animals/group. See table below.

| Distal | Control (no | | A9 + | |
|---|---|---|---|---|
| Organ | tumour cells) | A9 | IL-33 | TCl |
| Liver | 0.00% | 24.0-32.3% | 0.0-0.2% | 0.00% |
| Adrenal | 0.00% | 3.7-14.9% | 0.9-4.3% | 0.00% |
| glands | | | | |
| Lungs | 0.00% | 0.00-1.3% | 0.00-0.2% | 0.00% |
| Blood | 0.00% | Single cells 1-33 cells/ | 0.00-0.2% | 0.00% |
| | | 0.5 ml = 0.00% | | |
| Brain | 0.00% | 0.00% | 0.00% | 0.00% |

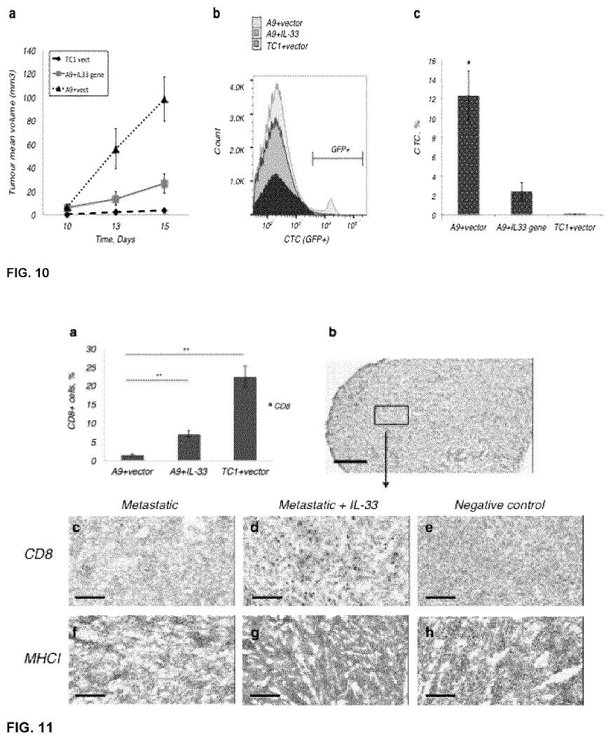

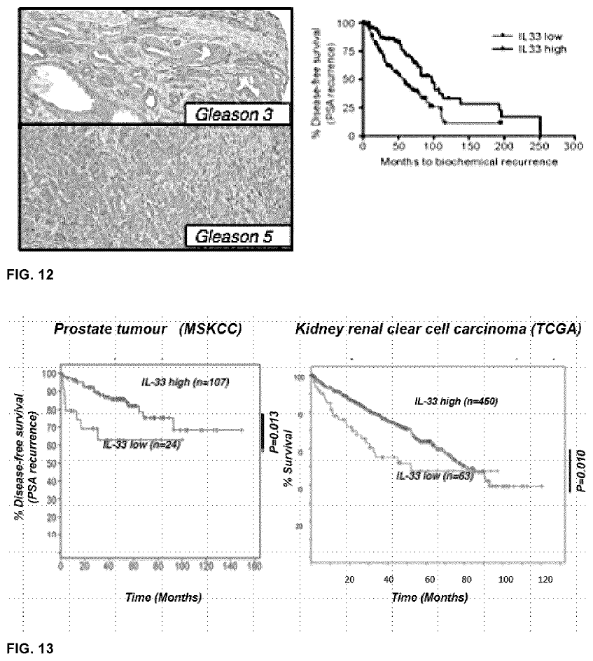

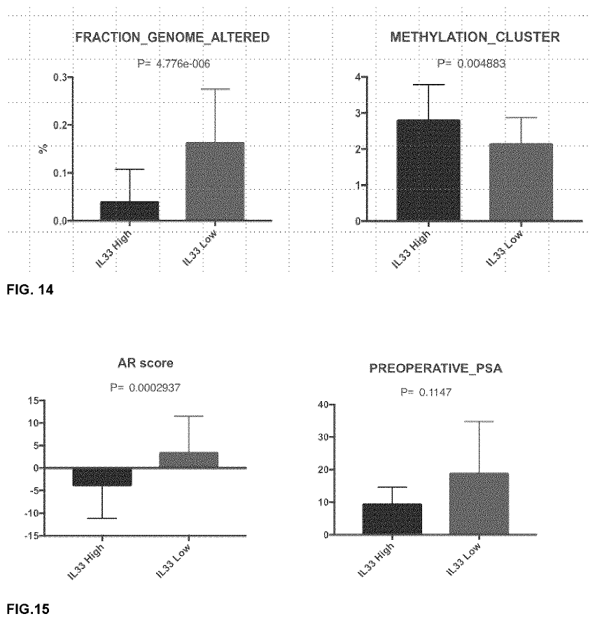

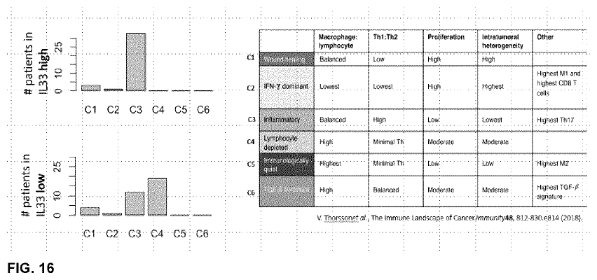

IL-33 gene-complementation suppresses tumour growth rate in vivo and inhibits metastatic spread of tumour cells in a mouse model. See FIG. 10.

### CONCLUSIONS

The preliminary analysis identifies candidate genes which potentially function as “master regulators” that regulate the IL-33 and APP pathways, affecting tumour outcome.

Potential inhibitory function imposed by FOXA1 on IL-33 and antigen presentation pathway components in prostate cancer was determined.

1. A method of inhibiting cancer progression, treating cancer and/or inhibiting metastatic spread of circulating tumor cells to distal organs, said method comprising enhancing expression and/or activity of interleukin-33 and/or APP by modulating one or more regulators of interleukin-33 and/or APP.

2. A method of stimulating antigen presentation or stimulating an immune response, said method comprising modulating expression and/or activity of one or more regulators of interleukin-33 and/or APP.

3. (canceled)

4. The method of claim 1, wherein said regulator is a negative regulator and said method comprises decreasing expression and or activity of said negative regulator.

5. The method of claim 1, wherein the one or more regulators are selected from the group consisting of FOXA1, KIF13A, MUC16, CSMD3, MUC17, ACACA, ATM, CTNNB1 and FBN3.

6. The method of claim 4, wherein said method comprises administration of one or more agents that target said one or more regulators and inhibits activity of said one or more regulators.

7. The method of claim 6, wherein said one or more agents are an antisense, antibody, aptamer or small molecule

8. The method of claim 7 wherein said one or more agents is used in combination with other therapies which stimulate immunity, treat cancer and/or inhibit cancer progression.

9. (canceled)

10. A method of distinguishing metastatic cells from non-metastatic cells, for determining progression to metastatic disease and/or determining prognosis of cancer, said method comprising determining expression and/or mutations of one or more master regulators involved in the IL33 and/or APP.

11. (canceled)

12. The method of claim 10, wherein the one or more master regulators are selected from the group consisting of FOX1A, KIF13A, MUC16, CSMD3, MUC17, ACACA, ATM, CTNNB1 and FBN3.

13. (canceled)

14. (canceled)

15. The method of claim 2, wherein said immune response is an anti-cancer or anti-pathogen immune response.
